(19) United States
(12) Patent Application Publication
(10) Pub. No.: US 2025/0118420 A1
(43) Pub. Date: Apr. 10, 2025

(54) MEDICAL SUPPORT DEVICE

(71) Applicant: NIHON KOHDEN CORPORATION, Tokyo (JP)

(72) Inventors: Takuya KAWASHIMA, Saitama (JP); Daisuke HORIGUCHI, Saitama (JP)

(21) Appl. No.: 18/897,717

(22) Filed: Sep. 26, 2024

(30) Foreign Application Priority Data
Oct. 6, 2023 (JP) 2023-174480

(51) Int. Cl. G16H 40/20; G16H 10/60; G16H 20/40

(52) U.S. Cl. CPC G16H 40/20; G16H 10/60; G16H 20/40

(57) ABSTRACT

A medical support device includes at least one processor, and a memory configured to store at least one instruction executable by the processor. When the at least one instruction is executed by the processor, the medical support device generates display data including expected procedure schedule information indicating an expected procedure schedule set for each of a plurality of procedures included in a surgery and progress information indicating progress of each of a plurality of procedures included in a current surgery.

### CROSS-REFERENCE TO RELATED APPLICATIONS

The present application claims the benefit of priority of Japanese Patent Application No. 2023-174480, filed on Oct. 6, 2023, the content of which is incorporated herein by reference.

### TECHNICAL FIELD

The presently disclosed subject matter relates to a medical support device.

### BACKGROUND ART

JP2023-002479A discloses a surgery support system that displays, on a monitor, a surgery schedule screen on which real-time surgery information is associated with a surgery schedule.

### SUMMARY

Incidentally, in many cases, a plurality of procedures are included in a surgery. However, while a schedule regarding a surgery in the related art includes a schedule regarding the entire surgery, there is room for improvement from a viewpoint of appropriately performing the entire surgery.

An object of the presently disclosed subject matter is to provide a medical support device capable of supporting appropriate advancement of each procedure included in a surgery.

According to a first aspect of the presently disclosed subject matter, there is provided a medical support device including:
at least one processor; and
a memory configured to store at least one instruction executable by the processor, in which
when the at least one instruction is executed by the processor, the medical support device
generates display data including expected procedure schedule information indicating an expected procedure schedule set for each of a plurality of procedures included in a surgery and progress information indicating progress of each of a plurality of procedures included in a current surgery.

With the medical support device according to the above-described configuration, the display data including the expected procedure schedule information indicating the expected procedure schedule set for each of the plurality of procedures included in the surgery and the progress information indicating the progress of each of the plurality of procedures included in the current surgery is generated. For example, a medical professional can confirm the progress of the plurality of procedures included in the current surgery while confirming the expected procedure schedule, by visually recognizing the display data. Therefore, with the medical support device according to the above-described configuration, it is possible to support appropriate advancement of a procedure included in a surgery.

According to the presently disclosed subject matter, it is possible to provide a medical support device capable of supporting appropriate advancement of each procedure included in a surgery.

### BRIEF DESCRIPTION OF DRAWINGS

FIG. 1 is a schematic view illustrating a medical support system according to an embodiment of the presently disclosed subject matter (hereinafter, also referred to as a present embodiment);

FIG. 2 is a diagram illustrating an example of past surgery information;

FIG. 3 is a diagram illustrating an example of patient information;

FIG. 4 is a diagram illustrating an example of medical professional information;

FIG. 5 is a flowchart illustrating a medical support method according to a first example;

FIG. 6 is a diagram illustrating an example of an image in which expected procedure schedule information is displayed;

FIG. 7 is a diagram illustrating an example of an image in which expected medication information is displayed;

FIG. 8 is a diagram illustrating an example of an image in which sub-expected surgery schedule information pertaining to each medical professional is displayed;

FIG. 9 is a flowchart illustrating a medical support method according to a second example;

FIG. 10 is a diagram illustrating an example of an image in which updated expected procedure schedule information is displayed;

FIG. 11 is a diagram illustrating an example of an image in which updated sub-expected surgery schedule information is displayed; and

FIG. 12 is a flowchart illustrating a notification method in a third example.

### DESCRIPTION OF EMBODIMENTS

Hereinafter, an example of an embodiment of the presently disclosed subject matter will be described with reference to the drawings.

### Overall Configuration

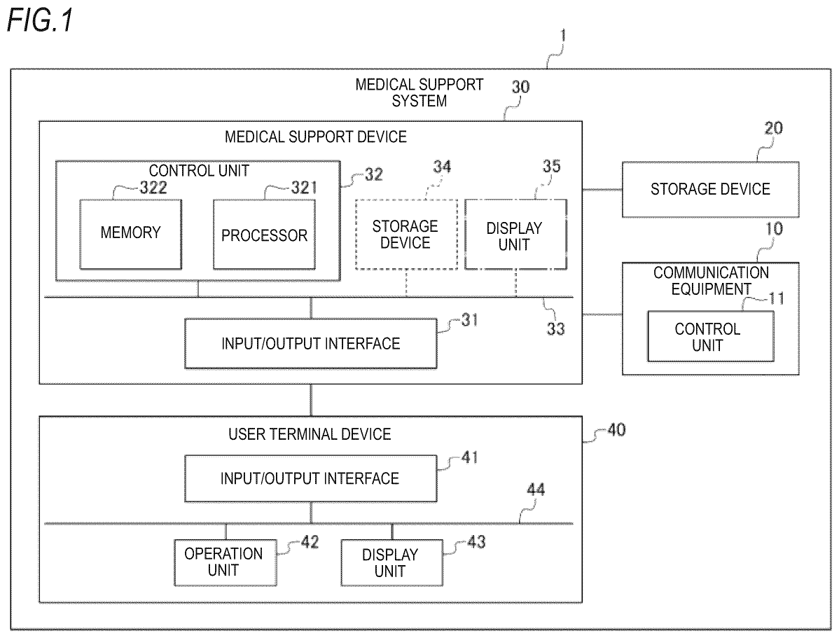

A medical support system 1 according to the present embodiment will be described with reference to FIGS. 1 to 4. FIG. 1 is a schematic view illustrating the medical support system 1 according to an embodiment of the presently disclosed subject matter. The medical support system 1 is used in, for example, a medical facility such as a hospital. In the present embodiment, it is assumed that percutaneous catheter ablation (an example of a surgery) was performed in the medical facility a plurality of times in the past. It is also assumed that percutaneous catheter ablation is being performed in the medical facility currently. In the present embodiment, it is assumed that the term “currently” means Aug. 1, 2023. As illustrated in FIG. 1, the medical support system 1 includes communication equipment 10, a storage device 20, a medical support device 30, and a user terminal device 40.

The communication equipment 10 is communicatively connected to the medical support device 30 in a wired or wireless manner. The communication equipment 10 is, for example, medical equipment such as a medical camera, a medical robot, information equipment with electronic medical records recorded therein, or an electric scalpel for use in a surgery or the like. The communication equipment 10 includes a control unit 11.

The control unit 11 includes, as a hardware configuration, a memory and a processor. The memory is configured with, for example, a read only memory (ROM) in which various computer programs and the like are stored or a random access memory (RAM) having a plurality of work areas in which various computer programs and the like that are executed by the processor are stored. The processor is, for example, a central processing unit (CPU), and is configured to load a designated computer program from various computer programs incorporated in the ROM to the RAM and execute various kinds of processing in cooperation with the RAM.

The control unit 11 acquires current procedure information regarding each of a plurality of procedures included in a current surgery. For example, when the communication equipment 10 is a medical camera, a medical robot, or information equipment having electronic medical records recorded therein, the control unit 11 acquires, as an example of the current procedure information, image data and video data captured during the surgery, medication data, biological information of the patient, and the like. For example, when the communication equipment 10 is an electric scalpel, the control unit 11 acquires, as the current procedure information, information regarding ON/OFF of a power supply (that is, information indicating whether the electric scalpel is used) or the like. The control unit 11 transmits the acquired current procedure information to the medical support device 30.

A plurality of procedures include in a surgery include various actions for performing the surgery. For example, a plurality of procedures included in a surgery include actions accompanying the surgery such as entrance and medical actions such as anesthesia induction. A plurality of procedures included in a surgery are set in advance for each surgery. Note that a plurality of procedures included in a surgery may be changed freely by, for example, a medical professional. For example, when a surgery is percutaneous catheter ablation, procedures included in the surgery are entrance, anesthesia induction, timeout, puncture, Brockenbrough, cauterization, left pulmonary vein (LPV) ablation, right pulmonary vein (RPV) ablation, provocation, sheath removal, and exit.

The current procedure information includes current medication information regarding medication administered to a patient in a procedure included in a current surgery and current transition information indicating transition of biological information of the patient in the procedure included in the current surgery. The control unit 11 can transmit the acquired current procedure information to the medical support device 30.

The control unit 11 can generate progress information indicating the progress of a procedure included in the current surgery based on the current procedure information. Here, the control unit 11 determines whether the procedure included in the current surgery is completed by analyzing image data, video data, or the like captured during the surgery and generates progress information based on the determination. When the control unit 11 generates the progress information, the control unit 11 transmits the generated progress information to the medical support device 30, for example, at a timing at which the procedure included in the current surgery is completed. Note that the control unit 11 may transmit the generated progress information to the medical support device 30 at other timings as well as the timing at which the procedure included in the current surgery is completed. That is, the control unit 11 transmits the generated progress information to the medical support device 30 at least at the timing at which the procedure included in the current surgery is completed.

The storage device 20 is configured to store, for example, various kinds of data. The storage device 20 is, for example, a storage device (storage) such as a hard disk drive (HDD), a solid state drive (SSD), or a flash memory. In the storage device 20, for example, past surgery information, patient information, and medical professional information are stored.

The past surgery information is information regarding a past surgery in a medical facility where the medical support system 1 is used. The past surgery information includes surgery day information, surgery name information, involved medical professional information, target patient information, past procedure information regarding each of a plurality of procedures included in the past surgery, past overall required time information regarding the past surgery, past assistance information, and the like.

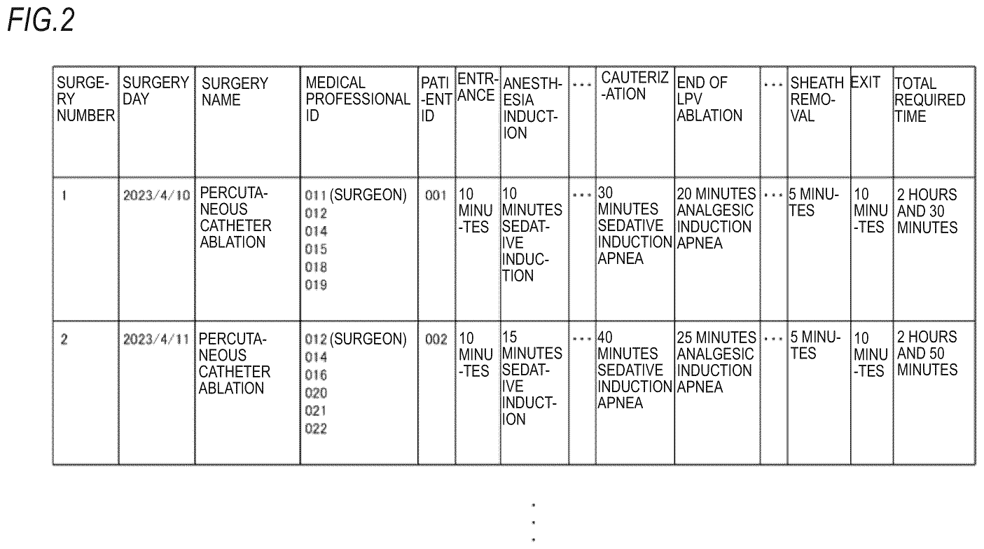

The surgery day information is information indicating a day on which the past surgery was performed. For example, a day on which a surgery with a surgery number 1 illustrated in FIG. 2 was performed is Apr. 10, 2023.

The surgery name information is information indicating a name of the past surgery. For example, a name of the surgery with the surgery number 1 illustrated in FIG. 2 is percutaneous catheter ablation.

The involved medical professional information is information regarding medical professionals involved in the past surgery. For example, medical professionals involved in the surgery with the surgery number 1 illustrated in FIG. 2 are medical professionals whose medical professional IDs are 011, 012, 014, 015, 018, and 019. The involved medical professional information is not limited to the medical professional ID, and may be, for example, a medical professional name. The involved medical professional information includes surgeon information indicating a surgeon in the surgery. For example, a surgeon in the surgery with the surgery number 1 illustrated in FIG. 2 is a medical professional whose medical professional ID is 011.

The target patient information is information indicating a patient in the past surgery. For example, a patient in the surgery with the surgery number 1 illustrated in FIG. 2 is a patient whose patient ID is 001. The target patient information is not limited to the patient ID, and may be, for example, a patient name.

The past procedure information includes past required time information and past order number regarding each of the plurality of procedures included in the past surgery. The past required time information is information indicating a required time of a procedure included in the past surgery. For example, the past required time information pertaining to the procedure of anesthesia induction in the surgery with the surgery number 1 illustrated in FIG. 2 is information indicating that the required time of the procedure was 10 minutes. The past order information is information indicating an order in which each procedure is performed in a surgery including a plurality of procedures. For example, the past order information pertaining to the procedure of anesthesia induction in the surgery with the surgery number 1 illustrated in FIG. 2 is information indicating that the order of the procedure was a second procedure in the entire surgery.

The past overall required time information is information indicating a required time of the overall past surgery. For example, the past overall required time information in the surgery with the surgery number 1 illustrated in FIG. 2 is information indicating that a time required for the surgery with the surgery number 1 was two hours and 30 minutes.

The past assistance information includes medication information regarding medication administered to the patient in the past surgery and transition information indicating transition of biological information of the patient in the past surgery. The medication information includes timing information, content information, dosage information, and ingredient information. The timing information is information indicating a timing of medication administered to the patient in the past surgery. The content information is information indicating the contents of medicine administered to the patient in the past surgery. The dosage information is information indicating the dosage of medicine administered to the patient in the past surgery. The ingredient information is information indicating the ingredients of medicine administered to the patient in the past surgery. In FIG. 2, for convenience of drawing, only a part of information in the past assistance information is illustrated.

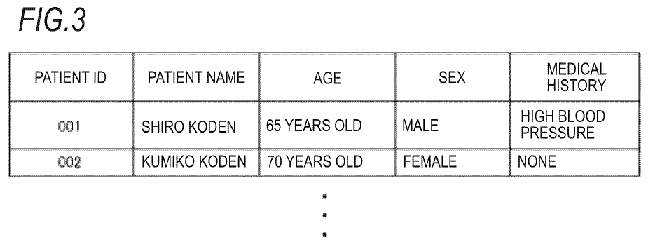

As illustrated in FIG. 3, the patient information includes age information indicating the age of the patient, sex information indicating the sex of the patient, and medical history information indicating the medical history of the patient. For example, the age information regarding SHIRO KODEN is information indicating that the age of SHIRO KODEN is 65 years old. For example, the sex information regarding SHIRO KODEN is information indicating that SHIRO KODEN is male. For example, the medical history information regarding SHIRO KODEN is information indicating that SHIRO KODEN has a medical history of high blood pressure.

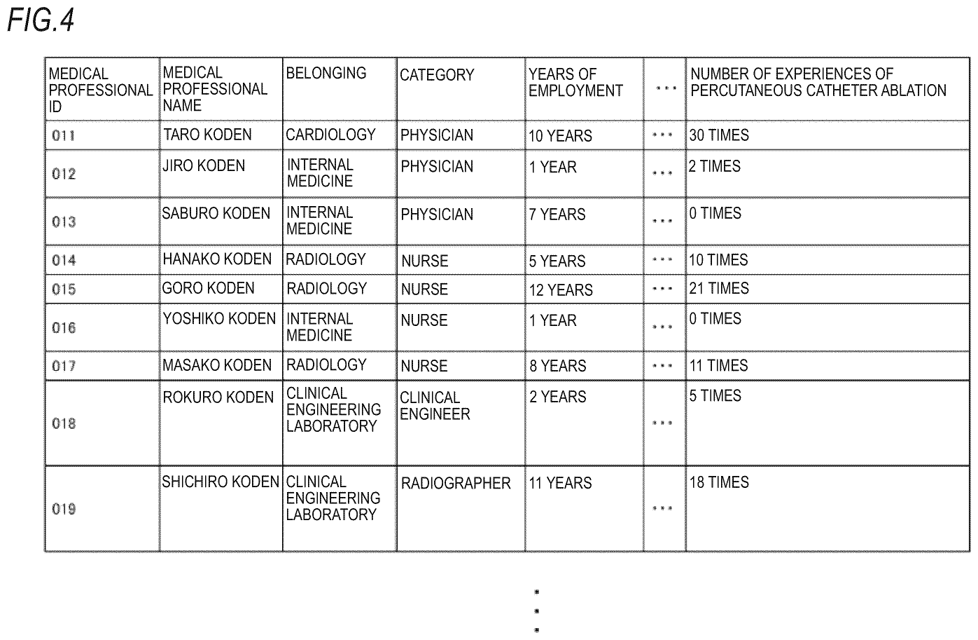

As illustrated in FIG. 4, the medical professional information includes belonging information indicating belonging of a medical professional, classification information indicating classification of a medical professional, and experience information regarding a surgery of a medical professional. The belonging of the medical professional is, for example, cardiology, internal medicine, or clinical engineering laboratory. The classification of the medical professional is, for example, physician or nurse. The experience information includes, for example, years-of-employment information indicating years of employment of a target medical professional, and number-of-experiences information indicating the number of experiences of a surgery of a target medical professional. For example, the belonging information regarding TARO KODEN is information indicating that the belonging of TARO KODEN is cardiology. For example, the classification information regarding TARO KODEN is information indicating that the classification of TARO KODEN is physician. For example, the years-of-employment information of TARO KODEN is information indicating that the years of employment of TARO KODEN is 10 years. For example, the number-of-experiences information regarding percutaneous catheter ablation of TARO KODEN is information indicating that TARO KODEN performed catheter ablation 30 times.

Returning to FIG. 1, the medical support device 30 will be described. The medical support device 30 is provided in, for example, a medical facility such as a hospital. The medical support device 30 is, for example, a biological information monitor device including a general server device and a display. When the medical support device 30 is a biological information monitor device, the medical support device 30 is disposed in, for example, a surgical room. When the medical support device 30 is a biological information monitor device, the medical support device 30 can display, for example, biological information (biological waveform information or the like) and information indicating the progress of a surgery (progress information or the like). Here, the medical support device 30 may be configured to switch display of biological information and display of information indicating the progress of the surgery. The medical support device 30 includes an input/output interface 31 and a control unit 32. The units are connected communicatively via a bus 33.

The input/output interface 31 functions as an interface of the communication equipment 10 and the medical support device 30. The input/output interface 31 also functions as an interface of the storage device 20 and the medical support device 30. The input/output interface 31 also functions as an interface of the medical support device 30 and the user terminal device 40.

The control unit 32 may have a hardware configuration similar to the control unit 11 of the communication equipment 10. The control unit 32 includes at least one processor 321, and a memory 322 that stores at least one instruction executable by the processor 321.

In the present embodiment, a computer-readable medium can be used. The computer-readable medium indicates any types of physical memory (RAM, ROM, or the like) that can store information or data readable by the processor. The computer-readable medium can store an instruction regarding processing to be executed by one or more processors. The term “computer-readable medium” includes a tangible article and excludes any carrier signal or any transitory signal (that is, indicating a non-transitory medium). Examples of the non-transitory computer-readable medium include a magnetic recording medium (for example, a flexible disk, a magnetic tape, or a hard disk drive), a magneto-optical recording medium (for example, a magneto-optical disk), a compact disc read only memory (CD-ROM), a CD-R, a CD-R/W, and a semiconductor memory (for example, a mask ROM, a programmable ROM (PROM), an erasable PROM (EPROM), or a flash ROM).

The control unit 32 acquires start time information indicating a surgery start time. For example, when a user inputs the start time information to the user terminal device 40, the input information is transmitted from the user terminal device 40 to the medical support device 30. As such, the medical support device 30 acquires the start time information.

The control unit 32 acquires at least one of the patient information and the medical professional information. For example, when the user inputs at least one of the patient information and the medical professional information to the user terminal device 40, the input information is transmitted from the user terminal device 40 to the medical support device 30. As such, the medical support device 30 acquires at least one of the patient information and the medical professional information.

The control unit 32 acquires the past surgery information from the storage device 20 according to, for example, a request signal from the user terminal device 40 described below. The control unit 32 acquires the past surgery information from the storage device 20 based on at least one of the patient information and the medical professional information. For example, when the control unit 32 acquires the past surgery information from the storage device 20 based on the patient information regarding SHIRO KODEN and the medical professional information regarding TARO KODEN, the control unit 32 acquires, from the storage device 20, the past surgery information pertaining to the surgery of which the patient is SHIRO KODEN and in which TARO KODEN is involved.

The control unit 32 generates expected surgery schedule information and expected assistance information based on the acquired past surgery information. The expected surgery schedule information is information obtained by statistical information processing on the past surgery information. The expected surgery schedule information includes expected procedure schedule information indicating an expected procedure schedule set for each of a plurality of procedures included in a surgery.

The expected procedure schedule information includes expected required time information indicating a required time of each procedure included in the current surgery, and order information indicating an order of each procedure included in the current surgery over the entire surgery. The expected required time information is generated by the control unit 32 based on the past required time information. In the present embodiment, the expected required time information is a median value of required times of the procedure included in the past surgery. Note that the expected required time information may be, for example, an average value of the required time of the procedure included in the past surgery. The control unit 32 generates expected overall required time information based on the expected required time information of each procedure. For example, the control unit 32 generates the expected overall required time information by totaling the required times indicated by the expected required time information of each procedure.

The expected assistance information includes at least one of expected medication information and expected transition information. The expected medication information includes expected timing information, expected content information, expected dosage information, and expected ingredient information. The expected timing information is information indicating a timing of medication scheduled to be administered to the patient in the current surgery. The expected content information is information indicating the contents of medicine scheduled to be administered to the patient in the current surgery. The expected dosage information is information indicating the dosage of medicine scheduled to be administered to the patient in the current surgery. The expected ingredient information is information indicating the ingredients of medicine scheduled to be administered to the patient in the current surgery.

The expected transition information is information indicating expected transition of the biological information of the patient in the current surgery. The expected transition information includes expected abnormality time information indicating a time at which a value indicated by the biological information of the patient becomes an abnormal value, and abnormality type information indicating a type of an abnormality of the patient at the time indicated by the expected abnormality time information. Examples of the type of the abnormality of the patient include apnea, bradycardia, and tachycardia. Apnea refers to a state in which breathing stops temporarily for ten seconds or more.

The control unit 32 can acquire a plurality of pieces of medical professional information indicating medical professionals involved in the surgery corresponding to the expected surgery schedule information and including classification information indicating the classifications of the medical professionals. Here, the control unit 32 generates sub-expected surgery schedule information of each medical professional based on the acquired classification information. The sub-expected surgery schedule information includes sub-expected procedure schedule information regarding each procedure of each medical professional. The sub-expected procedure schedule information includes sub-expected required time information and sub-order information. The sub-expected required time information is information indicating the required time of each procedure to be performed by a medical professional of a target classification among the procedures included in the current surgery. The sub-order information is information indicating an order of each procedure to be performed by the medical professional of the target classification among the procedures included in the current surgery.

When the control unit 11 of the communication equipment 10 generates the progress information, the control unit 32 acquires the current procedure information and the progress information or the progress information from the communication equipment 10. The control unit 32 acquires the progress information from the communication equipment 10, for example, at least at a timing at which a procedure included in the current surgery is completed. On the other hand, when the control unit 11 of the communication equipment 10 does not generate the progress information, the control unit 32 acquires the current procedure information from the communication equipment 10. The control unit 32 generates the progress information based on the acquired current procedure information. A generation method of the progress information that is performed by the control unit 32 can be similar to a generation method of the progress information that is performed by the control unit 11 of the communication equipment 10.

The control unit 32 generates display data including the expected surgery schedule information, the expected assistance information, the progress information, and the sub-expected surgery schedule information. The control unit 32 transmits the generated display data to the user terminal device 40.

The user terminal device 40 is a device that is operated by, for example, a medical professional such as a physician or a nurse. The user terminal device 40 is, for example, electronic equipment such as a desktop PC, a laptop PC, a tablet terminal, or a smartphone. The user terminal device 40 includes an input/output interface 41, an operation unit 42, and a display unit 43. The units are connected communicatively via a bus 44.

The input/output interface 41 functions as an interface of the medical support device 30 and the user terminal device 40.

The operation unit 42 is configured to receive an input operation of the user who operates the user terminal device 40, and generate a request signal corresponding to the input operation. The operation unit 42 is, for example, a touch panel disposed to be superimposed on the display unit 43, and operation buttons attached to a housing of the user terminal device 40. The operation unit 42 receives, for example, an input operation to acquire the display data and generates a request signal corresponding to the input operation. The generated request signal is transmitted to the medical support device 30 via the input/output interface 41. The medical support device 30 that receives the request signal generated by the operation unit 42 generates the display data based on the request signal and transmits the generated display data to the user terminal device 40.

The display unit 43 is, for example, a touch screen display such as a liquid crystal display or an organic EL display. The display unit 43 is configured to display, for example, an image based on the display data received from the medical support device 30.

### First Example

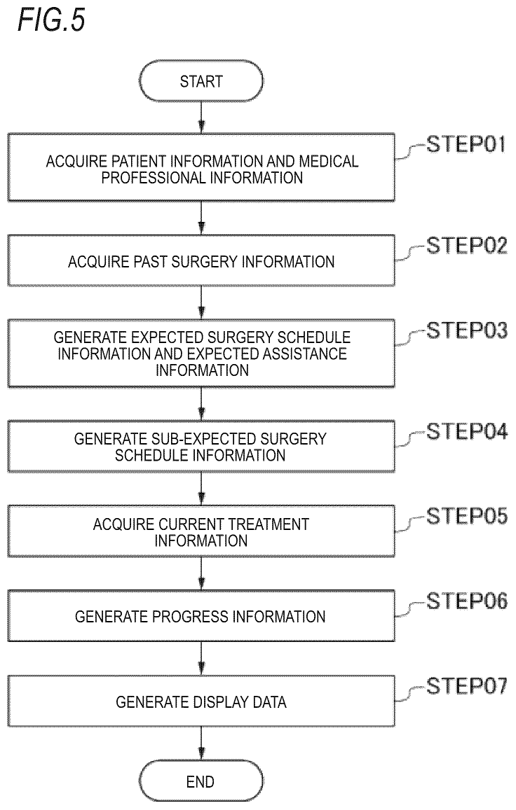

Next, a medical support method that is executed by the medical support device 30 in a first example will be described with reference to FIGS. 5 to 8. In the present example, it is assumed that percutaneous catheter ablation (an example of a surgery) is being performed from 9:00 on Aug. 1, 2023, and a current time is 9:32. In the surgery, the patient is SHIRO KODEN, and the medical professionals involved in the surgery are TARO KODEN, JIRO KODEN, HANAKO KODEN, YOSHIKO KODEN, ROKURO KODEN, and SHICHIRO KODEN. In the present example, it is assumed that the medical support device 30 acquires the current procedure information from the communication equipment 10 and generates the progress information based on the acquired current procedure information while the surgery is performed. The preconditions are similar in the following examples unless otherwise specifically described.

FIG. 5 is a flowchart illustrating the medical support method according to the first example. As illustrated in FIG. 5, the control unit 32 of the medical support device 30 acquires the patient information and the medical professional information (STEP01). In the present example, the user inputs the patient information and the medical professional information to the user terminal device 40 and the input patient information and medical professional information are transmitted from the user terminal device 40 to the medical support device 30, such that the control unit 32 acquires the patient information and the medical professional information. In the present example, the control unit 32 acquires the patient information regarding SHIRO KODEN and the medical professional information regarding TARO KODEN, JIRO KODEN, HANAKO KODEN, YOSHIKO KODEN, ROKURO KODEN, and SHICHIRO KODEN.

The control unit 32 acquires the past surgery information from the storage device 20 based on the acquired patient information and medical professional information (STEP02). In the present example, the control unit 32 acquires the past surgery information from the storage device 20 based on the patient information regarding SHIRO KODEN and the medical professional information regarding TARO KODEN. Note that the control unit 32 may acquire the past surgery information from the storage device 20 based on, for example, the patient information regarding SHIRO KODEN and the medical professional information regarding a plurality of medical professionals involved in the surgery. Since the control unit 32 acquires past surgery information from the storage device 20 based on the patient information regarding SHIRO KODEN and the medical professional information regarding TARO KODEN, the control unit 32 can acquire past surgery information from the storage device 20 considering the medical history and the like of SHIRO KODEN and the years of employment and the like of TARO KODEN.

The control unit 32 generates the expected surgery schedule information and the expected assistance information based on the acquired past surgery information (STEP03). In the present example, the control unit 32 generates the expected surgery schedule information and the expected assistance information based on the past surgery information pertaining to the surgery with the surgery number 1 and the like illustrated in FIG. 2.

As illustrated in FIG. 5, the control unit 32 generates the sub-expected surgery schedule information of each medical professional based on the acquired classification information (STEP04). In the present example, the control unit 32 generates the sub-expected surgery schedule information regarding TARO KODEN, JIRO KODEN, HANAKO KODEN, YOSHIKO KODEN, ROKURO KODEN, and SHICHIRO KODEN, respectively.

The control unit 32 acquires the current procedure information from the communication equipment 10 (STEP05). In the present example, the control unit 32 acquires the current procedure information from the communication equipment 10 at prescribed time intervals. The prescribed time interval is, for example, one-minute interval.

The control unit 32 generates the progress information based on the current procedure information acquired from the communication equipment 10 (STEP06). The control unit 32 generates the progress information by analyzing image data captured by the communication equipment 10 during the surgery, for example.

The control unit 32 generates the display data including the expected surgery schedule information, the expected assistance information, the progress information, the sub-expected surgery schedule information, the current medication information, and the current transition information (STEP07). When the display data is generated, the control unit 32 transmits the generated display data to the user terminal device 40.

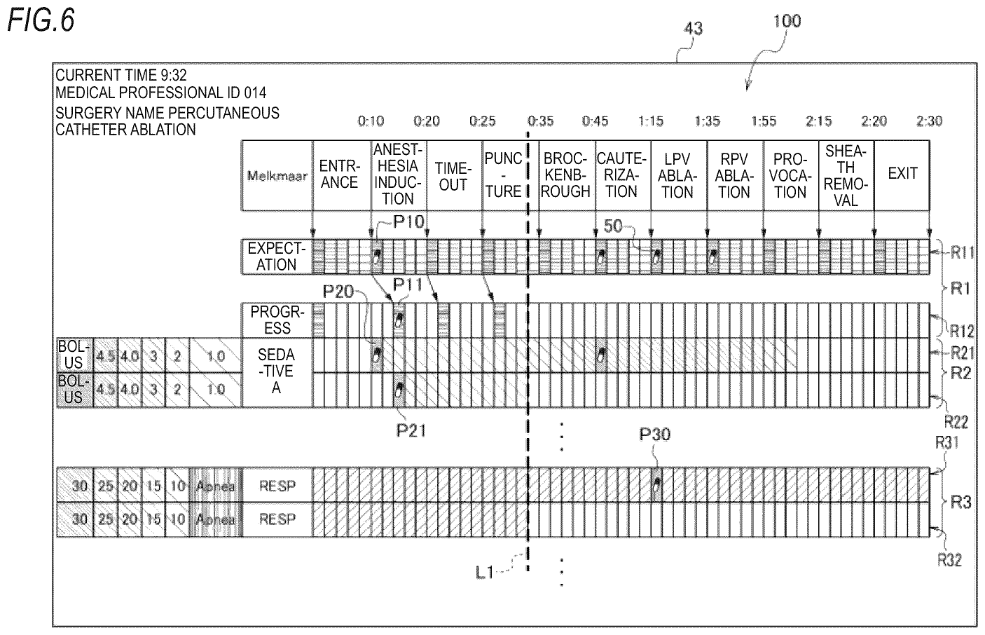

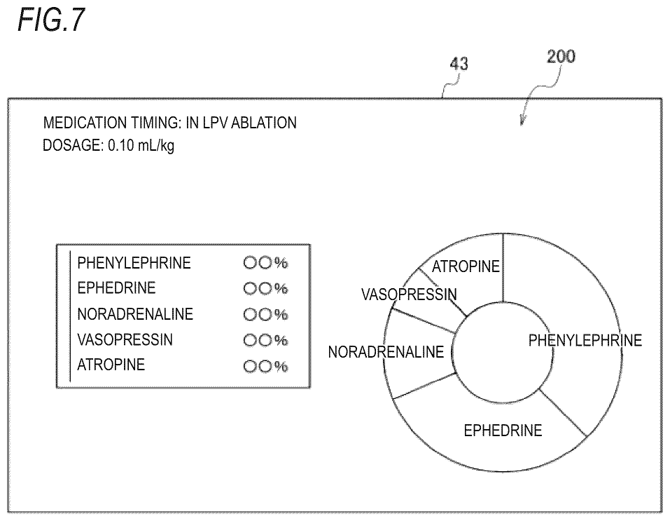

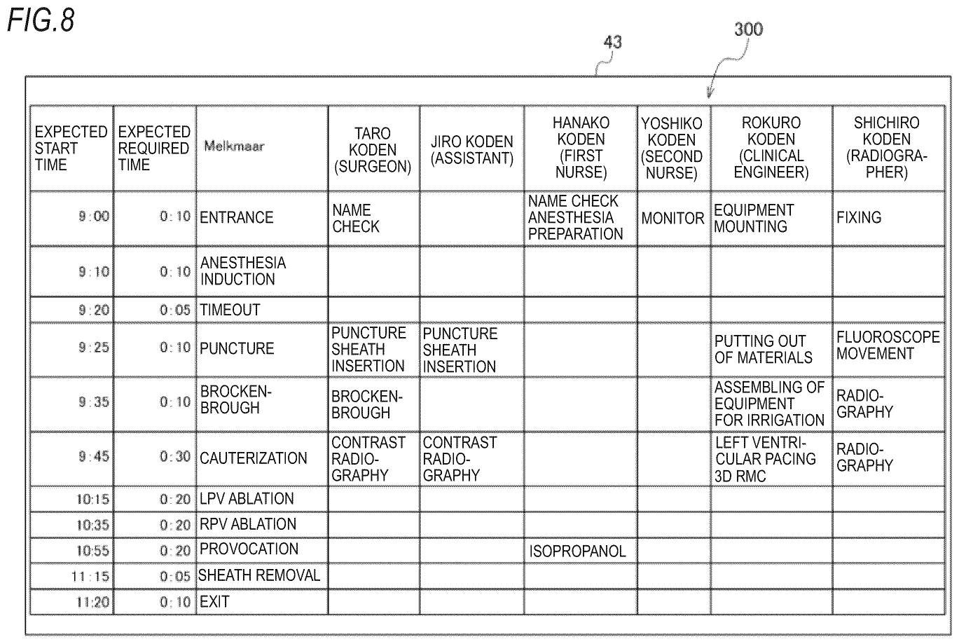

An image based on the display data received from the control unit 32 of the medical support device 30 is displayed on the display unit 43 of the user terminal device 40. Examples of the image displayed on the display unit 43 include an image 100 illustrated in FIG. 6, an image 200 illustrated in FIG. 7, and an image 300 illustrated in FIG. 8. Thereafter, the images 100, 200, and 300 will be described in connection with cases where the images 100, 200, and 300 are displayed on the display unit 43 of the user terminal device 40 that is operated by HANAKO KODEN, as an example. Images displayed on the display unit 43 of the user terminal device 40 that is operated by a medical professional other than HANAKO KODEN are basically the same as the images 100, 200, and 300 displayed on the display unit 43 of the user terminal device 40 that is operated by HANAKO KODEN.

A case where the image 100 illustrated in FIG. 6 is displayed on the display unit 43 will be described. In the image 100, a current time, a medical professional ID, a surgery name of a current surgery, a plurality of procedures included in the surgery, an expected start time of each procedure, the expected surgery schedule information, the progress information of each procedure, the expected medication information, the current medication information, the expected transition information, and the current transition information are displayed. The expected start time of each procedure is calculated by the control unit 32 based on, for example, the start time information, the order information, and the expected required time information.

In the image 100, a line L1 indicating a position of a current time is displayed. In the present example, the line L1 is a broken line, but may be a solid line, one-dot chain line, or the like. Methods for indicating the position of the current time is not limited to the line L1, and may be other figures, characters, or the like.

The image 100 includes a region R1, a region R2, and a region R3. In the region R1, the expected surgery schedule information including the expected procedure schedule information pertaining to each procedure and the progress information pertaining to each procedure are displayed.

The expected procedure schedule information and the progress information are displayed in two rows. That is, the expected procedure schedule information regarding the procedure linked to the progress information and included in the current surgery is displayed near the progress information. In the present example, the expected procedure schedule information is displayed above the progress information.

A shade of color in the region R1 represents a degree of deviation from a median value of a start time in each procedure. In the present specification, the shade of color is represented by the number of hatching lines. The more the hatching lines, the darker the color. In the present example, a darkest portion is a median value of the current time in the procedure, and the lighter the color is, the greater the deviation from the median value is. The darkest portion in an upper region (that is, a region representing an expected schedule of each procedure) R11 of the region R1 represents a position of a time corresponding to a median value of the expected start time of the procedure. For example, a portion P10 in FIG. 6 represents a position of a time corresponding to a median value of an expected start time in the procedure of anesthesia induction. A portion in a lower region (that is, a region representing the progress of each procedure) R12 of the region R1 with the same color as the darkest portion in the region R11 represents a position of a time at which the procedure is started. For example, a portion P11 in FIG. 6 represents a position of a time at which the procedure of anesthesia induction is started. As such, since a portion representing a position of a time corresponding to a median value of an expected start time in a certain procedure and a portion representing a position of a time at which the procedure is started have the same color, for example, it is possible for a medical professional to visually easily match the times to each other.

Expected medication information pertaining to sedative A is displayed in an upper region R21 of the region R2, and current medication information pertaining to the sedative A is displayed in a lower region R22 of the region R2, respectively. A shade of color in the region R2 represents the dosage of the sedative A to be administered to the patient, and the darker the color is, the greater the dosage of the sedative A is. The darkest portion in the upper region R21 of the region R2 represents a time at which the sedative A is scheduled to be bolus-administered. For example, a portion P20 in FIG. 6 represents a time at which the sedative A is scheduled to be bolus-administered in the procedure of anesthesia induction. The darkest portion in the lower region R22 of the region R2 represents a time at which the sedative A is bolus-administered to the patient. For example, a portion P21 in FIG. 6 represents a time at which the sedative A is bolus-administered to the patient in the procedure of anesthesia induction.

Expected transition information is displayed in an upper region R31 of the region R3, and current transition information is displayed in a lower region R32 of the region R3, respectively. A shade of color in the region R3 represents a condition level of the patient corresponding to a value indicated by the biological information of the patient. A vertically hatched portion (for example, a portion P30) indicates a time at which the value indicated by the biological information of the patient becomes an abnormal value. In the region R3, a type of an abnormality of the patient is also displayed at the time at which the value indicated by the biological information of the patient becomes the abnormal value. In the present example, as the type of the abnormality of the patient, apnea is displayed.

Next, the image 200 illustrated in FIG. 7 will be described. For example, when a medical professional clicks on an icon 50 included in the image 100 illustrated in FIG. 6, the image 200 illustrated in FIG. 7 is displayed on the display unit 43 of the user terminal device 40. In the present example, the icon 50 is an icon in the shape of a capsule of medicine, but may be icons of other types of design. Characters or the like may be displayed instead of the icon 50. As illustrated in FIG. 7, the image 200 includes expected timing information, expected dosage information, and expected ingredient information regarding medicine corresponding to the icon 50. Therefore, the medical professional can recognize a timing of medication pertaining to medicine corresponding to the icon 50 and the ingredients of the medicine by visually recognizing the image 200.

Next, the image 300 illustrated in FIG. 8 will be described. For example, when a medical professional performs a prescribed operation for displaying the image 300 on the display unit 43 on the operation unit 42 of the user terminal device 40, the image 300 is displayed on the display unit 43. The image 300 includes a sub-expected surgery schedule of each medical professional. The sub-expected surgery schedule is based on the sub-expected surgery schedule information pertaining to the corresponding medical professional. In the present example, the image 300 includes the sub-expected surgery schedules of TARO KODEN, JIRO KODEN, HANAKO KODEN, YOSHIKO KODEN, ROKURO KODEN, and SHICHIRO KODEN. Note that an image including the sub-expected surgery schedule of at least one of TARO KODEN, JIRO KODEN, HANAKO KODEN, YOSHIKO KODEN, ROKURO KODEN, and SHICHIRO KODEN may be displayed on the display unit 43. That is, the sub-expected surgery schedules of all medical professionals involved in the surgery do not need to be included in the image displayed on the display unit 43.

The medical professional can recognize the sub-expected surgery schedule of themselves or the sub-expected surgery schedules of other medical professionals by visually recognizing the image 300. For example, the medical professional can recognize a procedure scheduled to be performed by themselves in the current surgery or an expected start time of the procedure by visually recognizing the sub-expected surgery schedule of themselves.

With the medical support device 30 according to the above-described configuration, the display data including the expected procedure schedule information indicating the expected procedure schedule set for each of a plurality of procedures included in the surgery and the progress information indicating the progress of each of the plurality of procedures included in the surgery is generated. For example, the medical professional can recognize the progress of a plurality of procedures included in the current surgery while checking the expected procedure schedule by visually recognizing such display data. Therefore, with the medical support device 30, it is possible to support appropriate advancement of a procedure included in a surgery.

With the medical support device 30 according to the above-described configuration, since the expected procedure schedule information is based on the past procedure information regarding each of a plurality of procedures included in the past surgery, the expected procedure schedule indicated by the expected procedure schedule information has high accuracy.

With the medical support device 30 according to the above-described configuration, since the past procedure information is acquired based on the patient information and the medical professional information, the expected procedure schedule information generated based on the acquired past procedure information reflects the characteristics of the patient and the medical professional. Therefore, with the medical support device 30, it is possible to generate expected procedure schedule information with high accuracy.

With the medical support device 30 according to the above-described configuration, since the patient information includes the age information, the sex information, and the medical history information, it is possible to generate expected procedure schedule information with higher accuracy.

With the medical support device 30 according to the above-described configuration, since the medical professional information includes the belonging information, the classification information, and the experience information, it is possible to generate expected procedure schedule information with higher accuracy.

With the medical support device 30 according to the above-described configuration, since the expected procedure schedule information includes the expected required time information generated based on the past required time information, it is possible to ascertain a time required for each procedure.

With the medical support device 30 according to the above-described configuration, since the expected required time information is the medial value of the required time of the procedure included in the past surgery, it is possible to make the expected required time information as information indicating a more standardized expected required time.

With the medical support device 30 according to the above-described configuration, it is possible to generate the display data including the expected procedure schedule information and the progress information only by acquiring the current procedure information regarding each of the plurality of procedures included in the current surgery.

With the medical support device 30 according to the above-described configuration, even when the communication equipment 10 having a simple configuration is used, it is possible to generate the display data including the expected procedure schedule information and the progress information.

With the medical support device 30 according to the above-described configuration, the display data includes the sub-expected procedure schedule information of each medical professional generated based on the classification information indicating the classification of the medical professional. Therefore, it is possible for each medical professional to perform, for example, an appropriate procedure at an appropriate timing by visually recognizing the sub-expected procedure schedule information included in the display data.

With the medical support device 30 according to the above-described configuration, the expected procedure schedule information regarding the procedure linked to the progress information and included in the current surgery is displayed near the progress information. Therefore, it is possible for a medical professional to easily compare the expected procedure schedule information and the progress information using the display data generated by the medical support device 30.

### Second Example

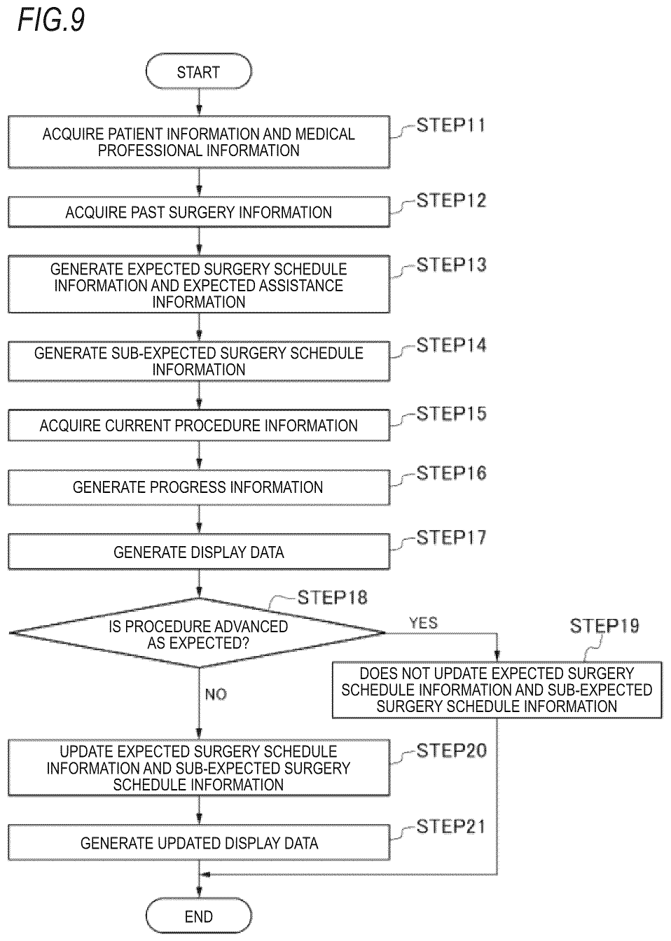

Next, a medical support method that is executed by the medical support device 30 in a second example will be described with reference to FIGS. 9 to 11. In the second example, portions similar to those in the first example are represented by the same reference signs as in the first example, and description of duplicate portions will not be repeated. In the present example, there is a difference from the first example in that the expected surgery schedule information and the sub-expected surgery schedule information are updated to reflect delay or hastening of each procedure performed until a current time among a plurality of procedures included in the surgery. In the present example, it is assumed that four-minute delay occurred in entrance, three-minute hastening occurred in anesthesia induction, and one-minute delay occurred in timeout.

In the present example, the control unit 32 can determine whether the current surgery is advanced as expected, based on the progress information and the expected procedure schedule information. The control unit 32 determines whether the current surgery is advanced as expected, based on a time at which determination is made that a current procedure is completed and an expected completion time calculated from the expected procedure schedule information and the start time information. The expected completion time is a time at which the procedure is estimated to be completed. When determination is made that the current surgery is advanced as expected, the control unit 32 does not update the expected surgery schedule information and the sub-expected surgery schedule information. On the other hand, when determination is made that the current surgery is not advanced as expected, the control unit 32 updates the expected surgery schedule information and the sub-expected surgery schedule information at a prescribed timing based on the progress information. In the present example, the control unit 32 updates the expected surgery schedule information and the sub-expected surgery schedule information when each procedure is completed. Note that the control unit 32 may update the expected surgery schedule information and the sub-expected surgery schedule information when each procedure is started or may update the expected surgery schedule information and the sub-expected surgery schedule information regularly at prescribed time intervals.

When the expected surgery schedule information and the sub-expected surgery schedule information are updated, the control unit 32 generates updated display data (an example of display data) based on the updated expected surgery schedule information and sub-expected surgery schedule information. The updated display data includes the updated expected surgery schedule information, the updated sub-expected surgery schedule information, the expected assistance information, the progress information, the current medication information, and the current transition information. The control unit 32 transmits the updated display data to the user terminal device 40.

Next, the medical support method that is executed by the medical support device 30 in the present example will be described with reference to FIG. 9. STEP11 to STEP17 are similar to STEP01 to STEP07 in the first example.

The control unit 32 determines whether the current surgery is advanced as expected based on the progress information and the expected procedure schedule information (STEP18). When determination is made that the current surgery is advanced as expected (in STEP18, YES), the control unit 32 does not update the expected surgery schedule information and the sub-expected surgery schedule information (STEP19). On the other hand, when determination is made that the current surgery is not advanced as expected (in STEP18, NO), the control unit 32 updates the expected surgery schedule information and the sub-expected surgery schedule information based on the progress information when each procedure is completed (STEP20).

In the present example, four-minute delay occurred in entrance, three-minute hastening occurred in anesthesia induction, and one-minute delays occurred in timeout. Therefore, in each procedure of entrance, anesthesia induction, and timeout, the control unit 32 updates the expected surgery schedule information and the sub-expected surgery schedule information based on the progress information pertaining to each procedure when each procedure is completed. Accordingly, delay or hastening in each procedure is reflected in the expected surgery schedule information and the sub-expected surgery schedule information.

When the expected surgery schedule information and the sub-expected surgery schedule information are updated, the control unit 32 generates the updated display data based on the updated expected surgery schedule information and sub-expected surgery schedule information (STEP21). When the updated display data is generated, the control unit 32 transmits the generated updated display data to the user terminal device 40.

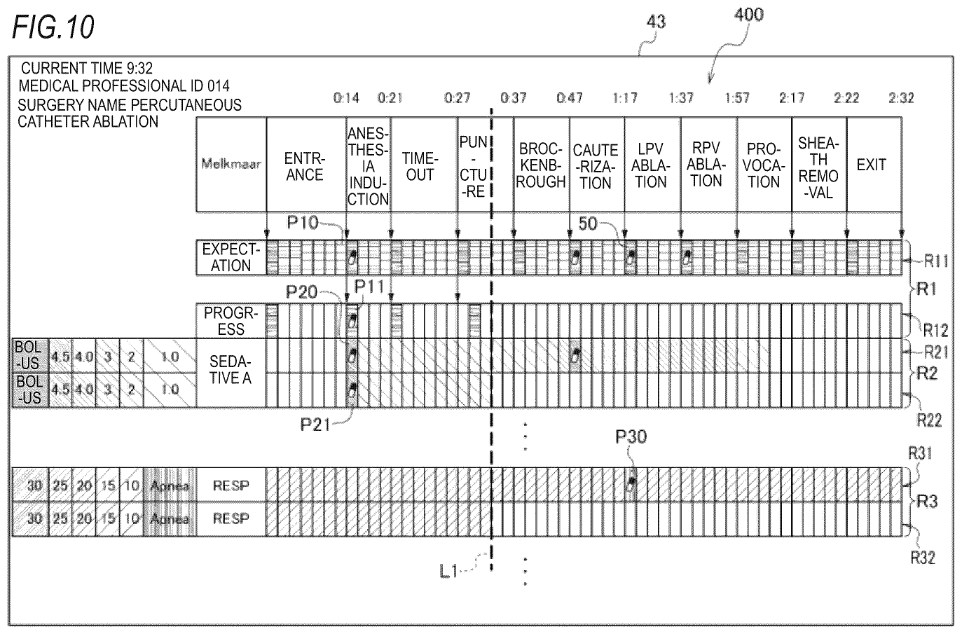

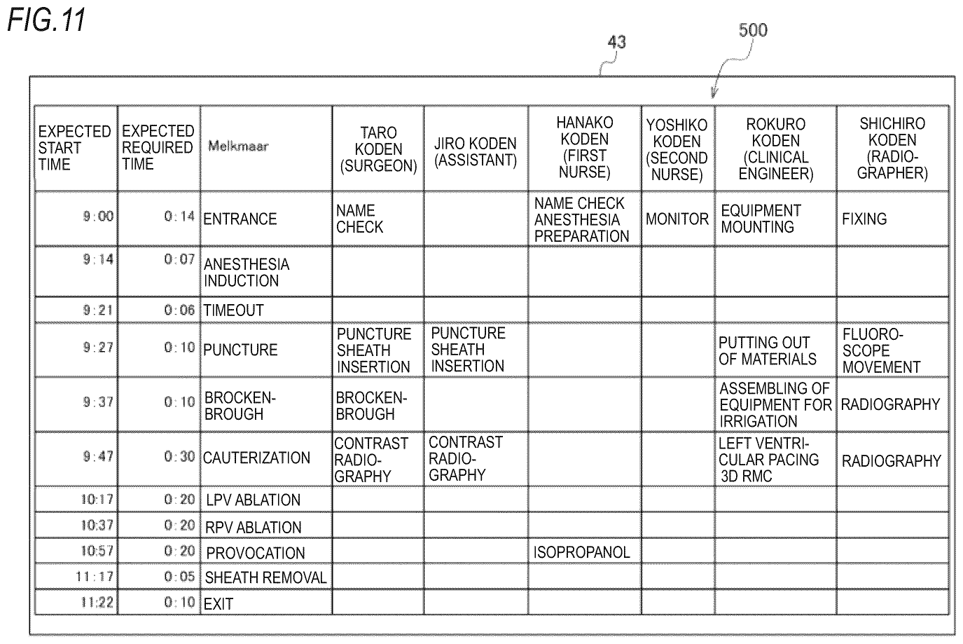

When the user terminal device 40 receives the updated display data from the medical support device 30, an image based on the updated display data is displayed on the display unit 43 of the user terminal device 40. Examples of the image displayed on the display unit 43 include the image 200 illustrated in FIG. 7, an image 400 illustrated in FIG. 10, and an image 500 illustrated in FIG. 11.

A case where the image 400 illustrated in FIG. 10 is displayed on the display unit 43 will be described. As illustrated in FIG. 10, in the image 400, a box is added or deleted based on delay or hastening that occurred until a current time in each procedure of entrance to puncture in the region R1 to the region R3. In the region R1 to the region R3, a completion time of each procedure of entrance to puncture is updated based on the delay or the hastening that occurred until the current time. The completion time is a time at which the procedure is completed. In the image 400, an expected start time of each procedure and an expected completion time of each procedure are updated based on a delay (in the present example, two-minute delay) that occurred until the current time in the procedures after Brockenbrough in the region R1 to the region R3. The image 400 is different from the image 100 according to the first example from such points. From other points, the image 400 is similar to the image 100 according to the first example.

Next, the image 500 illustrated in FIG. 11 will be described. For example, if the medical professional performs a prescribed operation for displaying the image 500 on the display unit 43 on the operation unit 42 of the user terminal device 40, the image 500 is displayed on the display unit 43. In the image 500, an updated sub-expected surgery schedule of each medical professional is included. The updated sub-expected surgery schedule is based on the updated sub-expected surgery schedule information pertaining to the corresponding medical professional. As illustrated in FIG. 11, the image 500 is different from the image 300 according to the first example in that the expected start time and the expected required time reflecting the delay or the hastening of each procedure performed until the current time among a plurality of procedures included in the surgery are included.

Also in the present example, the medical support device 30 obtains effects similar to the first example.

With the medical support device 30 according to the above-described configuration, the expected procedure schedule information is updated based on the progress information at a prescribed timing (in the present example, when each procedure is completed). For example, when a procedure included in the current surgery is delayed from an expected schedule, with the medical support device 30, it is possible to update the expected procedure schedule information considering the delay of the procedure. As such, with the medical support device 30, even if a procedure included in the current surgery is faster or slower than an expected schedule, it is possible to make the expected procedure schedule information as appropriate information.

### Third Example

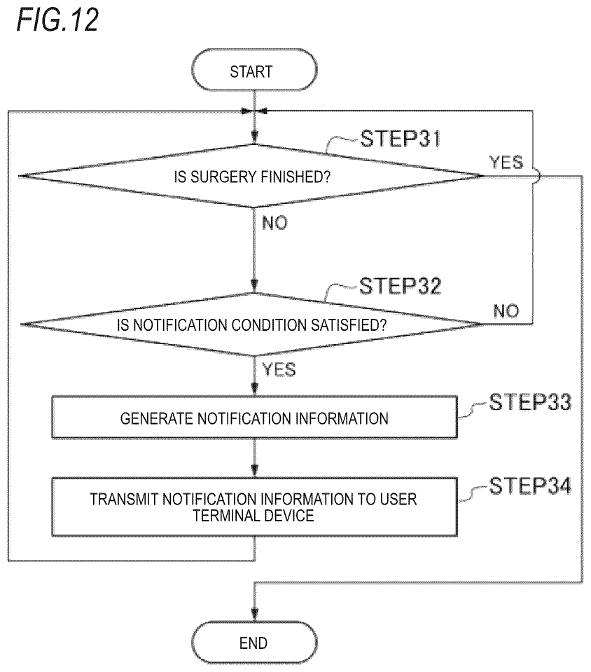

Next, a notification method that is executed by the medical support device 30 in a third example will be described with reference to FIG. 12. FIG. 12 is a flowchart illustrating the notification method in the third example. In the third example, portions similar to those in the first example or the second example are represented by the same reference signs as in the first example or the second example, and description of duplicate portions will not be repeated. The third example is different from the first example or the second example in that not only the medical support method executed by the medical support device 30 in the first example or the second example but also the notification method illustrated in FIG. 12 are executed. It is assumed that the notification method illustrated in FIG. 12 is executed in a period from the start to the end of the surgery.

In the present example, the control unit 32 can determine whether the current surgery is finished based on the progress information pertaining to a procedure of a last order among a plurality of procedures included in the current surgery.

The control unit 32 notifies of notification information for a medical professional scheduled to perform an upcoming procedure according to the progress of a procedure before the upcoming procedure. In the present example, the control unit 32 notifies of the notification information by determining whether a prescribed notification condition is satisfied, generating the notification information when determination is made that the notification condition is satisfied, and transmitting the generated notification information to the user terminal device 40. In the present example, the prescribed notification condition is a condition about whether a procedure two procedures before the upcoming procedure is completed. Note that the prescribed notification condition may be, for example, a condition about whether a procedure one procedure before the upcoming procedure is started. A notification method of the notification information may be an acoustic method or may be a visual method.

As illustrated in FIG. 12, the control unit 32 determines whether the current surgery is finished based on the progress information pertaining to the procedure of the last order among the plurality of procedures included in the current surgery (STEP31). When determination is made that the current surgery is finished (YES in STEP31), the control unit 32 ends processing pertaining to the notification method. On the other hand, when determination is made that the current surgery is not finished (NO in STEP31), the control unit 32 executes STEP32.

The control unit 32 determines whether the prescribed notification condition (that is, a condition about whether a procedure two procedures before the upcoming procedure is completed) is satisfied (STEP32). When determination is made that the prescribed notification condition is satisfied (YES in STEP32), the control unit 32 generates the notification information (STEP33). Then, the control unit 32 transmits the generated notification information to the user terminal device 40 (STEP34). When the user terminal device 40 receives the notification information from the control unit 32 of the medical support device 30, for example, character information based on the notification information is displayed on the display unit 43 of the user terminal device 40. The character information is, for example, a message “Your scheduled procedure is approaching”. The medical professional can recognize that their scheduled procedure is approaching by visually recognizing the character information displayed on the display unit 43 of the user terminal device 40. When STEP34 is executed, the control unit 32 executes STEP31 again. On the other hand, when determination is made that the prescribed notification condition is not satisfied (NO in STEP32), the control unit 32 does not execute STEP33 and STEP34, and executes STEP31 again.

As such, the control unit 32 executes the notification method illustrated in FIG. 12 from the start to the end of the surgery, and ends the execution of the notification method when the surgery ends.

Also in the present example, the medical support device 30 obtains effects similar to the first example and the second example.

With the medical support device 30 according to the above-described configuration, the notification information for the medical professional scheduled to perform the upcoming procedure is notified according to the progress of the procedure before the upcoming procedure (in the present example, a procedure two procedures before the upcoming procedure). Therefore, with the medical support device 30 according to the above-described configuration, it is possible for the medical professional to smoothly start their scheduled procedure.

The above-described embodiments are to make the presently disclosed subject matter easier to understand and do not to limit the presently disclosed subject matter. The presently disclosed subject matter can be changed and improved without departing from the spirit of the presently disclosed subject matter.

In the above-described embodiments, the control unit 32 may correct the expected procedure schedule information based on a total required time that is a total of the required times indicated by the past required time information and the required time of the entire past surgery. For example, when the total required time is three hours and the required time of the overall past surgery is two hours and 30 minutes, the control unit 32 multiplies the required time of each procedure included in the current surgery by ⅚ (150 minutes/180 minutes). That is, here, the control unit 32 corrects the expected procedure schedule information by multiplying the required time indicated by the expected required time information of each procedure by ⅚. With the medical support device 30 according to such a configuration, since the expected procedure schedule information is corrected based on the total required time and the required time of the overall past surgery, it is possible to make the expected procedure schedule information more appropriate.

In the above-described embodiment, as indicated by a broken line in FIG. 1, the medical support device 30 may include a storage device similar to the storage device 20. Here, the medical support system 1 may not include the storage device 20.

In the above-described embodiment, as indicated by a one-dot chain line in FIG. 1, the medical support device 30 may include a display unit 35 similar to the display unit 43 of the user terminal device 40. Here, the images 100, 200, 300, 400, and 500 may be displayed on the display unit 35 of the medical support device 30.

In the above-described embodiment, the control unit 32 generates the expected surgery schedule information and the expected assistance information based on the acquired past surgery information, but the presently disclosed subject matter is not limited thereto. The control unit 32 may generate expected surgery schedule information and expected assistance information based on, for example, schedule information indicating a schedule uniquely determined by a medical facility, a medical professional, or the like and assistance information indicating a timing or the like of medication uniquely determined by a medical facility, a medical professional, or the like. The control unit 32 may generate expected surgery schedule information and expected assistance information based on, for example, schedule information indicating a schedule recommended by a society in a medical field and assistance information indicating a timing or the like of medication recommended by the society in the medical field.

In the above-described embodiment, the past assistance information includes the medication information and the transition information, but the past assistance information may include only either of the medication information or the transition information.

In the above-described embodiment, the medication information includes the timing information, the content information, the dosage information, and the ingredient information, but the medication information only needs to include at least the timing information. The medication information may include at least one of the timing information, the content information, and the dosage information.

In the above-described embodiment, the medical professional information includes the belonging information, the classification information, and the experience information, but the medical professional information may not include all of such information. That is, the medical professional information may include at least one of the belonging information, the classification information, and the experience information.

In the above-described embodiment, the patient information includes the age information, the sex information, and the medical history information, but the patient information may not include all of such information. That is, the patient information may include at least one of the age information, the sex information, and the medical history information.

In the above-described embodiment, the control unit 32 of the medical support device 30 acquires the past surgery information from the storage device 20 based on the patient information and the medical professional information, but the presently disclosed subject matter is not limited thereto. For example, the control unit 32 may acquire the past surgery information from the storage device 20 based on either of the patient information or the medical professional information. The control unit 32 may acquire all past surgery information from the storage device 20.

In the above-described embodiment, the control unit 32 generates the display data including the expected surgery schedule information, the expected assistance information, the progress information, the sub-expected surgery schedule information, the current medication information, and the current transition information, but the presently disclosed subject matter is not limited thereto. For example, the display data may not include the expected assistance information or the progress information. The display data generated by the control unit 32 may not include at least one of the sub-expected surgery schedule information, the current medication information, and the current transition information.

In the above-described embodiment, the control unit 32 of the medical support device 30 generates the progress information, but for example, the control unit 11 of the communication equipment 10 may generate the progress information. Here, the communication equipment 10 transmits the progress information or the current procedure information and the progress information to the medical support device 30. Here, since the medical support device 30 acquires the progress information from the communication equipment 10, for example, a user such as a medical professional does not need to input the progress information to the medical support device 30, and as a result, it is possible to reduce burden on the user. Here, the medical support device 30 can acquire the progress information from the communication equipment 10, for example, at least at a timing at which the procedure included in the current surgery is completed. Here, since the progress information is acquired from the communication equipment 10 at least at the timing at which the procedure included in the current surgery is completed, it is possible to support appropriate advancement of a procedure included in a surgery with more excellent accuracy.

In the above-described embodiment, the medical support device 30 acquires the current procedure information from the communication equipment 10, but the presently disclosed subject matter is not limited thereto. For example, the user may input the current procedure information to the user terminal device 40 and the input current procedure information may be transmitted from the user terminal device 40 to the medical support device 30, such that the medical support device 30 acquires the current procedure information.

In the above-described embodiment, the expected procedure schedule information regarding the procedure linked to the progress information and included in the current surgery is displayed near the progress information, but the expected procedure schedule information may not be displayed near the progress information.

In the above-described embodiment, the expected transition information includes the expected abnormality time information, but the expected transition information may not include the expected abnormality time information. The expected transition information also includes the abnormality type information, but the expected transition information may not include the abnormality type information.

In the above-described embodiment, the image 100 or the image 400, the image 200, and the image 300 or the image 500 are displayed individually on the display unit 43 of the user terminal device 40, but the presently disclosed subject matter is not limited thereto. For example, two images (for example, the image 100 and the image 200) or three images (for example, the image 100, the image 200, and the image 300) among the images may be displayed simultaneously on the display unit 43. For example, when the user such as a medical professional taps a region of Melkmaar illustrated in FIG. 6, a pop-up or the like for selecting a medical professional is displayed. Then, when the user selects any medical professional from the displayed pop-up or the like, a sub-expected surgery schedule regarding the selected medical professional is inserted into the image 100 (for example, above Melkmaar illustrated in FIG. 6 or the like) and displayed.

In the third example, while the notification method of the notification information is a visual method, the notification method may be, for example, an acoustic method or an acoustic method and a visual method.

As described above, the following matters are disclosed in the present specification.

(1) A medical support device including:
at least one processor; and
a memory configured to store at least one instruction executable by the processor, in which
when the at least one instruction is executed by the processor, the medical support device
generates display data including expected procedure schedule information indicating an expected procedure schedule set for each of a plurality of procedures included in a surgery and progress information indicating progress of each of a plurality of procedures included in a current surgery.

(2) The medical support device according to (1), in which
past procedure information regarding each of a plurality of procedures included in a past surgery is acquired, and
the expected procedure schedule information is generated based on the past procedure information.

(3) The medical support device according to (2), in which
the past procedure information is acquired based on at least one of patient information indicating a patient of the current surgery and medical professional information indicating a medical professional involved in the current surgery.

(4) The medical support device according to (3), in which
the patient information includes at least one of age information indicating an age of the patient, sex information indicating a sex of the patient, and medical history information indicating a medical history of the patient.

(5) The medical support device according to (3) or (4), in which
the medical professional information includes at least one of belonging information indicating a belonging of the medical professional, classification information indicating a classification of the medical professional, and experience information of the medical professional regarding the current surgery. (6) The medical support device according to any one of (2) to (5), in which
the past procedure information includes past required time information indicating a required time of each procedure included in the past surgery,
expected required time information indicating a required time of each procedure included in the current surgery is generated based on the past required time information, and
the expected procedure schedule information includes the expected required time information.

(7) The medical support device according to (6), in which
the expected required time information is a median value of required times of the procedure included in the past surgery.

(8) The medical support device according to (7), in which
past overall required time information indicating a required time of the overall past surgery is further acquired, and
the expected procedure schedule information is corrected based on a total required time that is a total of the required times indicated by the past required time information, and the required time of the overall past surgery.

(9) The medical support device according to any one of (1) to (8), in which
the progress information is acquired from communication equipment configured to be communicable with the medical support device.

(10) The medical support device according to (9), in which
the progress information is acquired from the communication equipment at least at a timing at which the procedure included in the current surgery is completed.

(11) The medical support device according to any one of (1) to (10), in which
current procedure information regarding each of the plurality of procedures included in the current surgery is acquired, and,
the progress information is generated based on the current procedure information.

(12) The medical support device according to (11), in which
the current procedure information is acquired from communication equipment configured to be communicable with the medical support device.

(13) The medical support device according to any one of (1) to (12), in which
a plurality of pieces of the medical professional information indicating a medical professional involved in the current surgery and including classification information indicating a classification of the medical professional is acquired,
sub-expected procedure schedule information of each medical professional is generated based on the classification information, and
the display data includes the sub-expected procedure schedule information.

(14) The medical support device according to any one of (1) to (13), in which
notification information for a medical professional scheduled to perform an upcoming procedure is notified according to progress of a procedure before the upcoming procedure.

(15) The medical support device according to any one of (1) to (14), in which
the expected procedure schedule information regarding the procedure linked to the progress information and included in the current surgery is displayed near the progress information.

(16) The medical support device according to any one of (1) to (15), in which
the expected procedure schedule information is updated based on the progress information at a prescribed timing.

What is claimed is:

1. A medical support device comprising:
at least one processor; and
a memory configured to store at least one instruction executable by the processor, wherein
when the at least one instruction is executed by the processor, the medical support device
generates display data including expected procedure schedule information indicating an expected procedure schedule set for each of a plurality of procedures included in a surgery and progress information indicating progress of each of a plurality of procedures included in a current surgery.

2. The medical support device according to claim 1, wherein
past procedure information regarding each of a plurality of procedures included in a past surgery is acquired, and
the expected procedure schedule information is generated based on the past procedure information.

3. The medical support device according to claim 2, wherein
the past procedure information is acquired based on at least one of patient information indicating a patient of the current surgery and medical professional information indicating a medical professional involved in the current surgery.

4. The medical support device according to claim 3, wherein
the patient information includes at least one of age information indicating an age of the patient, sex information indicating a sex of the patient, and medical history information indicating a medical history of the patient.

5. The medical support device according to claim 3, wherein
the medical professional information includes at least one of belonging information indicating a belonging of the medical professional, classification information indicating a classification of the medical professional, and experience information of the medical professional regarding the current surgery.

6. The medical support device according to claim 2, wherein
the past procedure information includes past required time information indicating a required time of each procedure included in the past surgery,
expected required time information indicating a required time of each procedure included in the current surgery is generated based on the past required time information, and
the expected procedure schedule information includes the expected required time information.

7. The medical support device according to claim 6, wherein
the expected required time information is a median value of required times of the procedure included in the past surgery.

8. The medical support device according to claim 7, wherein
past overall required time information indicating a required time of the overall past surgery is further acquired, and
the expected procedure schedule information is corrected based on a total required time that is a total of the required times indicated by the past required time information, and the required time of the overall past surgery.

9. The medical support device according to claim 1, wherein
the progress information is acquired from communication equipment configured to be communicable with the medical support device.

10. The medical support device according to claim 9, wherein
the progress information is acquired from the communication equipment at least at a timing at which the procedure included in the current surgery is completed.

11. The medical support device according to claim 1, wherein
current procedure information regarding each of the plurality of procedures included in the current surgery is acquired, and
the progress information is generated based on the current procedure information.

12. The medical support device according to claim 11, wherein
the current procedure information is acquired from communication equipment configured to be communicable with the medical support device.

13. The medical support device according to claim 1, wherein
a plurality of pieces of the medical professional information indicating a medical professional involved in the current surgery and including classification information indicating a classification of the medical professional is acquired,
sub-expected procedure schedule information of each medical professional is generated based on the classification information, and
the display data includes the sub-expected procedure schedule information.

14. The medical support device according to claim 1, wherein notification information for a medical professional scheduled to perform an upcoming procedure is notified according to progress of a procedure before the upcoming procedure.

15. The medical support device according to claim 1, wherein
the expected procedure schedule information regarding the procedure linked to the progress information and included in the current surgery is displayed near the progress information.

16. The medical support device according to claim 1, wherein
the expected procedure schedule information is updated based on the progress information at a prescribed timing.
